(19) United States
(12) Patent Application Publication
(10) Pub. No.: US 2017/0151177 A1
(43) Pub. Date: Jun. 1, 2017

(54) LOXOPROFEN AND GAMMA-AMINOBUTIRIC ACID RECEPTOR AGONIST COMBINATIONS

(71) Applicant: SANOVEL ILAC SANAYI VE TICARET ANONIM SIRKETI, ISTANBUL (TR)

(72) Inventors: ÜMIT CIFTER, ISTANBUL (TR); ALI TÜRKYILMAZ, ISTANBUL (TR); YELDA ERDEM, ISTANBUL (TR); SEVAL ATAMAN, ISTANBUL (TR); AYSE ILDES ERDEM, ISTANBUL (TR); GAYE RAMAZANOGLU, ISTANBUL (TR)

(21) Appl. No.: 15/318,573

(22) PCT Filed: Jun. 29, 2015
(86) PCT No.: PCT/EP2015/064690

§ 371 Date: Dec. 13, 2016

(30) Foreign Application Priority Data
Jun. 30, 2014 (TR) 2014/07604

(51) Int. Cl. A61K 9/24; A61K 31/704; A61K 9/20; A61K 31/192

(52) U.S. Cl. CPC A61K 9/209; A61K 31/192; A61K 31/704; A61K 9/2054; A61K 9/2018; A61K 9/2013; A61K 9/2009; A61K 9/2059

(57) ABSTRACT

This invention is a novel pharmaceutical composition comprising loxoprofen or a pharmaceutically acceptable salt thereof in combination with gamma-aminobutiric acid receptor agonist or a pharmaceutically acceptable salt thereof with anti-inflammatory, analgesic and myorelaxant activity.

### TECHNICAL FIELD OF THE INVENTION

This invention is a novel pharmaceutical composition comprising loxoprofen or a pharmaceutically acceptable salt thereof in combination with gamma-aminobutiric acid receptor agonist or a pharmaceutically acceptable salt thereof with anti-inflammatory, analgesic and myorelaxant activity.

### BACKGROUND OF THE INVENTION

Loxoprofen is a non-steroidal anti-inflammatory drug in the propionic acid derivatives group. It is a prodrug and it is quickly converted to its active trans-alcohol metabolite following oral administration. It is a non-selective cyclooxygenase inhibitor and works by reducing the synthesis of prostaglandins from arachidonic acid. Its chemical name is (RS)-2-{4-[(2-oxocyclopentyl)methyl]phenyl}propanoic acid and its chemical structure is shown in the Formula I.

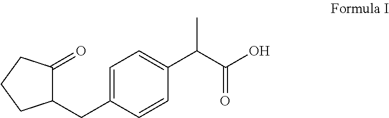 [SMILES: CC(C(=O)O)c1ccc(CC2CCCC2=O)cc1]

The patent application U.S. Pat. No. 4,161,538 (A) discloses the loxoprofen molecule.

The patent EP0947584 (B1) discloses an anti-inflammatory analgesic patch comprising loxoprofen or pharmaceutically acceptable salt thereof, water, crotamiton and a water soluble polymer.

The patent application WO0247661 (A1) discloses pharmaceutical composition for intramuscular injection containing loxoprofen or a pharmaceutically acceptable salt thereof, as an active ingredient.

The patent EP1806152 (B1) discloses an external preparation containing a pharmacologically active component that is loxoprofen and a lipophilic polyglycerin fatty acid ester.

The use of 3-demethyl-thiocolchicine glucoside, known as thiocolchicoside, is also widespread in therapy for treating contractures and inflammatory conditions that affect the muscular system. Thiocolchicoside, has been claimed to possess GABA-mimetic and glycinergic actions, in other way we can say that thiocolchicoside is a gamma-aminobutiric acid receptor agonist. Its chemical structure is shown in Formula 2.

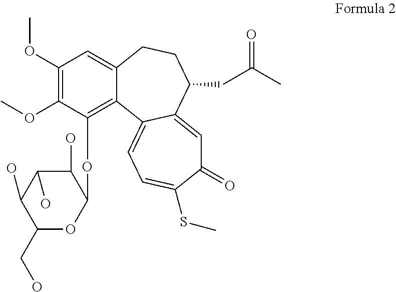

It has recently been shown that thiocoichicoside's activity can be ascribed to its ability of interacting with the strychnine-sensitive glycine receptors and therefore that compounds endowed with glycino-mimetic activity can be used in the rheumatologic-orthopedic field for their muscle relaxant properties.

The usual initial dose is 8 mg daily by mouth and maximum recommended oral dose is 16 mg. It has also been given intramuscularly, in doses up to 8 mg daily, or applied as cream or ointment (Sean C Sweetman, Martindale The Complete Drug Reference, thirty-fifth edition 2007, Vol. 1, page 1738).

Gamma-aminobutiric acid receptor agonists suitable for use in the context of the present invention other than thiocolchicoside are baclofen or muscimol or a pharmaceutically acceptable salt thereof.

Chemical name of Baclofen is 4-amino-3-(4-chlorophenyl) butanoic acid and the structural formula is shown in Formula 3:

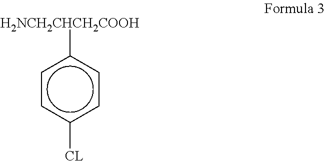

Marketed product of baclofen tablets contain 10 mg or 20 mg baclofen, for oral administration,

Chemical name of muscimol is 3-Hydroxy-5-aminomethylisoxazole and the structural formula is shown in Formula 4:

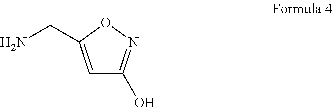

Gamma-aminobutiric acid receptor agonists have been evaluated alone or in combination with conventional analgesics for the treatment of pain. Mixed and unpredictable results have been obtained in a pharmaceutical composition. But loxoprofen has not previously been combined with gamma-aminobutiric acid receptor agonists, in particular with thiocolchicoside in a pharmaceutical composition for the treatment of inflammatory, pain and musculoskeletal diseases.

Thiocolchicoside is a known gamma-aminobutiric acid receptor agonists agent used in the treatment of painful muscle spasms or spasticity occurring in musculoskeletal and neuromuscular disorders and for treating contractures and inflammatory conditions that affect the muscular system.

European patent EP 0 837 684 B1 (Sanofi-Synthelabo) 13 Jun. 1995, relates to pharmaceutical compositions containing, in solid form, a diclofenac salt and thiocolchicoside combined with at least one pharmaceutically acceptable carrier are provided for use in therapy.

It is well known that drugs used in the same therapeutic area or even for treating the same indication cannot always be combined a priori with the expectation of at least additive therapeutic effects. The scientific literature is full of examples wherein compounds of different classes, which are used to treat the same indications, cannot be combined into safe and efficacious dosage forms thereby resulting in incompatible drug combinations. The reasons for this unexpected lack of compatibility are varied; however, it is often found that the incompatible drug combinations result in increased side effects, unwanted drug interactions or new side effects. More specifically, in the area of analgesia there are drug combinations that are contraindicated for some or all of these very same reasons.

Conventional analgesic and myorelaxant therapy generally involves administration of a pharmaceutical composition containing one or more different analgesic and muscle relaxant drugs. However, not all combinations of analgesic drugs and muscle relaxant drugs are more suitable, in terms of safety or efficacy, than the administration of a single product.

To date no pharmaceutical compositions or dosage forms comprising a combination of a loxoprofen and thiocolchicoside, have been made.

### DETAILED DESCRIPTION OF THE INVENTION

This invention is a pharmaceutical composition comprising loxoprofen or a pharmaceutically acceptable salt thereof in combination with thiocolchicoside or a pharmaceutically acceptable salt thereof with anti-inflammatory, analgesic and myorelaxant activity administrated oral, parenteral, intramuscular and topical in tablet, bilayer tablet, multi layer tablet, capsule, injectable preparat, suspension, syrup, sachet, ointment, cream or gel form.

Novel pharmaceutical composition in the form of a tablet or a capsule administrated orally may provide a significant advance in the available treatments. Such combination therapy may also provide for therapeutic improvements owing to the potential synergistic effect provided by the combination.

Therefore, further aspects of the present invention concern the use of pharmaceutical composition comprising loxoprofen in combination with thiocolchicoside for use in the treatment of painful muscle spasms associated with static and functional disorders of vertebra or occurred in post-operations of osteoarthritis, pain and inflammatory symptoms associated with tissue trauma, degenerative vertebra diseases as torticollis, dorsalgia, lombalgia, disk hernia, neurologic and traumatic disorders associated with spasticity.

As mentioned above, this invention comprising active ingredient, loxoprofen or a pharmaceutically acceptable salt thereof in combination with thiocolchicoside or a pharmaceutically acceptable salt thereof.

The main challenges when combining two or more molecules in the same pharmaceutical form are (a) to guarantee the physicochemical compatibility between the different active ingredients and/or between the active ingredients and the excipients used; and (b) to insure the therapeutical compatibility between the two active ingredients regarding their pharmacokinetic and/or pharmaceutical properties in order that the posology of the combined composition allows to obtain safe and efficient plasma levels of both pharmacological agents.

According to main challenges mentioned above, the pharmaceutical composition comprising loxoprofen in combination with thiocolchicoside have an additive analgesic effect in relief of postoperative pain and provide greater analgesia with the results in a lower incidence of side effects according to priori. These pharmaceutical combinations are administrated orally, parenterally, intramuscularly and topically.

The pharmaceutical compositions of the invention include tablets, capsules, injectables, suspensions, syrups, sachets, ointments, creams or gels can be made in accordance with methods that are standard in the art. Examples of oral dosage forms include tablets (including compressed, coated or uncoated), capsules, hard or soft gelatin capsules, pellets, pills, powders, granules, elixirs, tinctures, colloidal dispersions, dispersions, effervescent compositions, films, sterile solutions or suspensions, syrups or emulsions and the like.

Preferably, the combination of a loxoprofen with thiocolchicoside will be in the form of a conventional tablet or capsule. And it may be granulated by methods such as, dry granulation, low- or high-shear granulation, wet granulation or fluidized-bed granulation. Low-shear granulation, high-shear granulation, wet granulation and fluidized-bed granulation generally produce harder, less friable tablets.

This invention is a pharmaceutical composition, wherein the loxoprofen or a pharmaceutically acceptable salt and thiocolchicoside or a pharmaceutically acceptable salt are combined together with at least one pharmaceutically acceptable excipient.

Gamma-aminobutiric acid receptor agonists suitable for use in the context of the present invention are selected from the group comprising thiocolchicoside, baclofen, muscimol, acamprosate, methaqualone, picamilon, progabide, tiagabine, lesogaberan and phenibut or a pharmaceutically acceptable salt thereof. Preferably, the gamma-aminobutiric acid receptor agonist is a thiocolchicoside, baclofen or musimol or a pharmaceutically acceptable salt thereof, more preferably it is thiocolchicoside or a pharmaceutically acceptable salt thereof.

As mentioned above, this invention comprising active ingredient, loxoprofen or a pharmaceutically acceptable salt thereof in combination with thiocolchicoside wherein the loxoprofen is present in an amount of between 10.0% and 40.0% and the thiocolchicoside is present in an amount of 0.5% and 10.0% (w/w), preferred embodiments an amount of the loxoprofen is between 20.0% and 30.0% and the thiocolchicoside is between 1.0% and 5.0% (w/w).

As mentioned above, this invention comprises the combination of loxoprofen with thiocolchicoside and at least one pharmaceutically acceptable excipient. Suitable pharmaceutically acceptable excipients comprise but are not limited to disintegrants, fillers, binders, glidants and lubricants or mixtures thereof.

In a preferred embodiment of the present invention, said disintegrants comprise, but are not limited to microcrystalline cellulose, low-substituted hydroxypropyl cellulose, alginic acid and alginates, ion-exchange resins, magnesium aluminum silica, sodium carboxy methyl cellulose, carboxy methyl cellulose calcium, polyvinylpyrrolidone, docusate sodium, guar gum, polacrilin potasium, poloxomer, sodium alginate, sodium glysin carbonate, or the mixtures thereof, preferably, it is microcrystalline cellulose.

In a preferred embodiment of the present invention, said fillers comprise, but are not limited to lactose monohydrate, dibasic calcium phosphate, tribasic calcium phosphate, sorbitol, sucrose, trehalose, isomalt, microcrystalline cellulose, mannitol, starch, sodium carbonate, sodium bicarbonate, dextrose, maltodextrine, calcium carbonate, xylitol or the mixtures thereof, preferably, it is lactose monohydrate.

In a preferred embodiment of the present invention, said binders comprise, but are not limited to pregelatinised starch, hydroxypropyl cellulose, sugars, glycose syrups, natural gums, guar gum, gelatins, pullulan, polymetacrylates, collagen, agar, algynate, sodium alginate, hyaluronic acid, pectin, tragacanth gum, carboxymethyl cellulose, polyvinylpyrrolidone, polyethylene glycol, polyvinyl alcohol, polyvinyl acetate and their copolymers, hydroxypropyl methyl cellulose, carboxy methyl cellulose, methyl cellulose, microcrystalline cellulose, polyvinylalcohol, carrageenan, carbomer, poloxamer, polyacrylamide, aluminum hydroxide, benthonite, laponite, setostearyl alcohol, polyoxyethylene-alkyl ethers, acacia mucilage, polydextrose, polyethylene oxide or the mixtures thereof, preferably, it is pregelatinised starch

In a preferred embodiment of the present invention, said lubricants comprise, but are not limited to magnesium stearate, sodium stearyl fumarate, sodium lauryl sulphate, magnesium lauryl sulphate, fumaric acid, glyceryl palmitostearate, hydrogenated natural oils, zinc stearate, calcium stearate, silica, talc, stearic acid, polyethylene glycol, paraffin or the mixtures thereof, preferably, it is magnesium stearate.

In a preferred embodiment of the present invention, said glidants comprise, but are not limited to colloidal silicon dioxide, stearic acid, talk, aluminium silicate or the mixtures thereof, preferably, it is colloidal silicon dioxide.

The invention is further defined by reference to the following example. Although the example is not intended to limit the scope of the present invention, it should be considered in the light of the description detailed above.

### Example 1

Loxoprofen + Thiocolchicoside

% Amount (w/w)

Internal phase

| | |
|---|---|
| loxoprofen sodium hydrate | 20.0-30.0% |
| thiocolchicoside | 1.0-5.0% |
| lactose monohydrate | 5%-50% |
| pregelatinized starch | 1%-20% |
| microcrystalline celulose | 5%-50% |

External phase

| | |
|---|---|
| magnesium stearate | 0.1%-5% |
| colloidal silicon dioxide | 0.05%-2% |

Process of Example 1:

Loxoprofen sodium hydrate, thiocolchicoside, lactose monohydrate, pregelatinized starch and microcrystalline cellulose are sieved and mixed. After obtaining the homogenous mixture, wet granulation process is applied with water and then dried in an oven at 55° C. The obtained granul is then sieved and colloidal silicon dioxide and magnesium stearate is added respectively and mixed. Then the powder mixture is pressed into tablets weighing 250 mg. These tablets preferably coated by a coating material including conventional coating polymers like Opadry®.

In other preferred embodiment, these powder mixtures are filled in a capsule by capsule filling machine to obtain conventional capsule forms in appropriate length.

In other preferred embodiments of this invention, the solid dosage form is a bilayer tablet having the loxoprofen in one layer and gamma-aminobutiric acid receptor agonists which are thiocolchicoside or baclofen or muscimol particular thiocolchicoside in another layer. The amount of loxoprofen employed in such bilayer tablets preferably ranges from 10.0% to 40.0%, and more preferably is 20.0% to 30.0% (w/w). The amount of thiocolchicoside employed in such bilayer tablets preferably ranges from 2 mg to 20 mg and more preferably is 4 mg or 16 mg.

This invention is further defined by reference to the following example. Although the example is not intended to limit the scope of the present invention, it should be considered in the light of the description detailed above.

### Example 2 (Bilayer)

Loxoprofen Granules:

Loxoprofen granules are granulated with high shear granulator and at last they are sieved and dried by fluid bed drier.

| Content | % amount (w/w) |
|---|---|
| Loxoprofen | 20.0-30.0 |
| Lactose | 20.0-60.0 |
| Microcrystalline cellulose | 9.00-27.0 |
| Croscarmellose sodium | 1.25-3.75 |
| Hydroxypropyl cellulose | 1.75-5.25 |
| Colloidal silicon dioxide | 0.25-0.75 |
| Magnesium stearate | 0.25-0.75 |

Thiocolchicoside Granules:

Thiocolchicosiede granules are granulated with high shear granulator and at last they are sieved and dried by fluid bed drier.

| Content | % amount (w/w) |
|---|---|
| Thiocolchicoside | 1.00-5.00 |
| Lactose | 37.5-112.5 |
| Starch | 7.50-22.5 |
| Gelatine | 0.75-2.25 |
| Sucrose | 1.50-4.50 |
| Talc | 1.00-3.00 |
| Magnesium stearate | 0.50-1.50 |

The solid dosage form mentioned above is a bilayer tablet having the loxoprofen granules in one layer and thiocolchicoside granules in second layer. These granules are compressed by 2 layered tablet press machine to obtain bilayer tablet forms and these bilayer tablets preferably covered by a coating material including conventional coating polymers like Opadry II (HP)®.

1. A pharmaceutical composition comprising loxoprofen or a pharmaceutically acceptable salt thereof in combination with gamma-aminobutiric acid receptor agonist or a pharmaceutically acceptable salt thereof.

2. The pharmaceutical composition according to claim 1, wherein the gamma-aminobutiric acid receptor agonist is selected from the group comprising thiocolchicoside, baclofen, muscimol, acamprosate, methaqualone, picamilon, progabide, tiagabine, lesogaberan and phenibut or a pharmaceutically acceptable salt thereof.

3. The pharmaceutical composition according to claim 2, wherein the gamma-aminobutiric acid receptor agonist is thiocolchicoside or baclofen or musimol or a pharmaceutically acceptable salt thereof.

4. The pharmaceutical composition according to claim 3, wherein the gamma-aminobutiric acid receptor agonist is thiocolchicoside or a pharmaceutically acceptable salt thereof.

5. The pharmaceutical composition according to claim 4, wherein the loxoprofen or a pharmaceutically acceptable salt thereof is present in an amount of between 10.0% and 40.0% (w/w) and thiocolchicoside or a pharmaceutically acceptable salt thereof is present in an amount of 0.5% and 10.0% (w/w).

6. The pharmaceutical composition according to claim 5, wherein the loxoprofen or a pharmaceutically acceptable salt thereof is present in an amount of between 20.0% and 30.0% (w/w) and the thiocolchicoside or a pharmaceutically acceptable salt thereof is present in an amount of between 1.0% and 5.0% (w/w).

7. The pharmaceutical composition according to any preceding claims, wherein the loxoprofen or a pharmaceutically acceptable salt thereof and thiocolchicoside or a pharmaceutically acceptable salt thereof are combined together with at least one pharmaceutically acceptable excipient.

8. The pharmaceutical composition according to claim 1, wherein at least one pharmaceutically acceptable excipient is selected from a group comprising disintegrants, fillers, binders, glidants and lubricants or mixtures thereof.

9. The pharmaceutical composition according to any preceding claims, wherein said pharmaceutical composition is administrated orally, parenterally, intramuscularly or topically.

10. The pharmaceutical composition according to any preceding claims, wherein said pharmaceutical composition is formulated as a tablet, bilayer tablet, multilayer tablet, capsule, sachet, injectable preparat, suspension, syrup, gel, cream or ointment.

11. The pharmaceutical composition according to claim 10, wherein said pharmaceutical composition is in the form of a tablet or a bilayer tablet or a capsule.

12. The pharmaceutical composition according to claim 11, wherein said pharmaceutical composition is in the form of a tablet or a capsule.

13. The pharmaceutical composition according to claim 12, comprising
% Amount (w/w)
a) Internal phase

| | |
|---|---|
| i. loxoprofen sodium hydrate | 20.0-30.0% |
| ii. thiocolchicoside | 1.0-5.0% |
| iii. lactose monohydrate | 5%-50% |
| iv. pregelatinized starch | 1%-20% |
| v. microcrystalline celulose | 5%-50% |

b) External phase

| | |
|---|---|
| i. magnesium stearate | 0.1%-5% |
| ii. colloidal silicon dioxide | 0.05%-2% |

14. The pharmaceutical composition according to claim 11, wherein said pharmaceutical composition is in the form of a bilayer tablet.

15. The pharmaceutical composition according to claim 14, wherein said bilayer tablet having the loxoprofen or a pharmaceutically acceptable salt thereof in one layer and gamma-aminobutiric acid receptor agonists in another layer.

16. The pharmaceutical composition according to claim 15, wherein said bilayer tablet having the loxoprofen in one layer and thiocolchicoside or baclofen or muscimol in other layer.

17. The pharmaceutical composition according to claim 16, wherein said bilayer tablet having the loxoprofen or a pharmaceutically acceptable salt thereof in one layer and the thiocolchicoside or a pharmaceutically acceptable salt thereof in another layer.

18. The pharmaceutical composition according to any preceding claim, for use in the treatment of painful muscle spasms associated with static and functional disorders of vertebra or occurred in post-operations of osteoarthritis, pain and inflammatory symptoms associated with tissue trauma, degenerative vertebra diseases as torticollis, dorsalgy, lombalgy, disk hernia, neurologic and traumatic disorders associated with spasticity.
